(19) United States
(12) Patent Application Publication
(10) Pub. No.: US 2026/0118323 A1
(43) Pub. Date: Apr. 30, 2026

(54) METHOD FOR DETERMINING MULTI-CHARACTERISTIC COMPONENTS IN ANSHEN DINGZHI WAN

(71) Applicant: Anhui University of Chinese Medicine, Hefei (CN)

(72) Inventors: Caiyun ZHANG, Hefei (CN); Chengjie XING, Hefei (CN); Yu GAO, Hefei (CN); Pengfei XIE, Hefei (CN); Xiaoxiao SHAN, Hefei (CN); Can PENG, Hefei (CN); Wei ZHANG, Hefei (CN); Huanhuan LIU, Hefei (CN); Xiaoli WANG, Hefei (CN); Chuanshan JIN, Hefei (CN)

(73) Assignee: Anhui University of Chinese Medicine, Hefei (CN)

(21) Appl. No.: 19/066,195

(22) Filed: Feb. 28, 2025

(30) Foreign Application Priority Data
Oct. 28, 2024 (CN) 202411511996.2

(51) Int. Cl. G01N 30/06; G01N 30/02; G01N 30/74; G01N 33/00

(52) U.S. Cl. CPC G01N 30/06; G01N 30/74; G01N 33/0098; G01N 2030/027

(57) ABSTRACT

A method for determining multi-characteristic components of ASDZW is provided. By optimizing the gradient elution program of high performance liquid chromatography (HPLC), strictly controlling the initial mobile phase ratio of the gradient elution program, cooperating with the selections of the chromatographic column and the mobile phase, the invention finally realizes the simultaneous, rapid and comprehensive determination of six characteristic components in ASDZW under the condition of a single wavelength; specifically, the six characteristic components are polygalaxanthone III, 3,6′-disinapoyl sucrose, ginseng saponin Rb1, β-asarone, dehydrotumulosic acid, and pachymic acid. The whole detection process is a single wavelength detection with high detection efficiency and short analysis time, and the detection limits of the six components are lower than 0.008 μg/mL, meaning this method has high sensitivity, good specificity, accurate results, and good reproducibility.

### CROSS REFERENCE TO THE RELATED APPLICATIONS

This application is based upon and claims priority to Chinese Patent Application No. 202411511996.2, filed on Oct. 28, 2024, the entire contents of which are incorporated herein by reference.

### TECHNICAL FIELD

The invention belongs to the field of traditional Chinese medicine composition analysis technology, especially relates to a method for determining multi-characteristic components of Anshen Dingzhi Wan (ASDZW).

### BACKGROUND

Anshen Dingzhi Wan (ASDZW) is one of the classic prescriptions recorded in Medical Mind Comprehension, which has the effects of tranquilizing the mind, benefiting qi and calming down the heart. It is mainly used in the treatment of anxiety, depression, insomnia and other diseases in clinics. It is composed of poria, poria with hostwood, panax ginseng, polygala tenuifolia, acori tatarinowii rhizoma, dragon teeth, and cinnabaris. According to the analysis of basic theory of traditional Chinese medicine, poria and poria with hostwood in this prescription are sweet, light and flat, and benefit the four meridians of heart, lung, spleen and kidney, which can calm and tranquilize the mind and mood; Panax ginseng has a great nourishment of renal qi; acori tatarinowii rhizoma is pungent, bitter and warm, and benefits the meridians to the heart and stomachwhich can eliminate phlegm, induce resuscitation, awaken consciousness, and improve intelligence; polygala tenuifolia is pungent, bitter and warm, and benefits the three meridians of heart, kidney and lung, which has the effects of tranquilizing the mind, eliminating phlegm and detumescence; polygala tenuifolia combined with acori tatarinowii rhizoma can eliminate phlegm, induce resuscitation and benefit the mind; the dragon teeth and cinnabaris are used to calm down the mind; and all drugs in ASDZW prescription work together to calm the mind and tranquilize the nerves. Modern studies have shown that ASDZW has a variety of pharmacological activities, such as reducing the level of inflammatory factors, inhibiting inflammatory response, reducing neuronal damage in rats with Alzheimer's disease, repairing brain learning and memory function, and also has a good effect on depression, anxiety, and primary insomnia.

ASDZW is composed of a variety of traditional Chinese medicines, with complex components that are difficult to control and conduct comprehensive quality evaluations. Liquid chromatography is a common method in the study of quality control of traditional Chinese medicine, but how to quickly and accurately determine the content of various characteristic components in ASDZW by liquid chromatography has not been disclosed.

### SUMMARY

In view of the above problems, according to the prescription composition of Anshen Dingzhi Wan (ASDZW), this invention adopts a high performance liquid chromatography (HPLC) with diode array detection (DAD) detector to simultaneously determine the content of six characteristic components of Polygalaxanthone III, 3,6′-disinapoyl sucrose, ginseng saponin Rb1, β-asarone, dehydrotumulosic acid and pachymic acid in ASDZW within 90 min at 203 nm.

The invention proposes a method for determining multi-characteristic components of ASDZW, including the following steps:
preparing a test solution of ASDZW;
weighing polygalaxanthone III, 3,6′-disinapoyl sucrose, ginseng saponin Rb1, β-asarone, dehydrotumulosic acid, and pachymic acid to prepare a mixed reference solution;
detecting the test solution and the mixed reference solution by HPLC to obtain liquid chromatograms of the test solution and a liquid chromatography of the mixed reference solution, respectively;
comparing the liquid chromatogram of the test solution with the liquid chromatogram of the mixed reference solution, and determining characteristic peaks and contents of multi-characteristic components in ASDZW according to a comparison result.

Furthermore, preparing the test solution of ASDZW, including:
performing an ultrasonic treatment for ASDZW in water-saturated n-butanol solution, taking a filtrate after filtrating and evaporating to dryness; redissolving a dried filtrate with methanol and filtering by a microporous filter membrane to obtain the test solution of ASDZW.

Furthermore, a ratio of a mass of ASDZW to a volume of water-saturated n-butanol solution is 1: (15-20) g/mL; specifically, the ratio can be 1:15 g/mL, 1:16 g/mL, 1:17 g/mL, 1:19 g/mL, 1:19 g/mL, 1:20 g/mL; an ultrasonic treatment time is 20-40 min; exemplarily, the ultrasonic treatment time can be 20 min, 25 min, 28 min, 320 min, 32 min, 35 min, 40 min; a pore size of a microporous filter membrane is 0.22-0.45 μm; exemplarily, the pore size can be 0.22 μm, 0.25 μm, 0.30 μm, 0.45 μm.

Furthermore, preparing the mixed reference solution, including:
accurately weighting reference substances of polygalaxanthone III, 3,6′-disinapoyl sucrose, ginseng saponin Rb1, β-asarone, dehydrotumulosic acid, and pachymic acid, dissolving with methanol, and diluting to prepare a reference solution.

Furthermore, a concentration of polygalaxanthone III in the mixed reference solution is 0.01-0.32 mg/mL, for example, the concentration can be 0.01 mg/mL, 0.07 mg/mL, 0.18 mg/mL, 0.20 mg/mL, 0.26 mg/mL, 0.32 mg/mL;
a concentration of 3,6′-disinapoyl sucrose is 0.03-1.00 mg/mL, for example, the concentration can be 0.03 mg/mL, 0.10 mg/mL, 0.18 mg/mL, 0.25 mg/mL, 0.5 mg/mL, 1.00 mg/mL;
a concentration of ginseng saponin Rb1 is 0.02-0.60 mg/mL, for example, the concentration can be 0.02 mg/mL, 0.10 mg/mL, 0.17 mg/mL, 0.25 mg/mL, 0.35 mg/mL, 0.55 mg/mL, 0.60 mg/mL.
a concentration of β-asarone is 0.02-0.76 mg/mL, for example, the concentration can be 0.02 mg/mL, 0.10 mg/mL, 0.17 mg/mL, 0.25 mg/mL, 0.35 mg/mL, 0.60 mg/mL, 0.76 mg/mL;
a concentration of dehydrotumulosic acid is 0.01-0.16 mg/mL, for example, the concentration can be 0.01 mg/mL, 0.03 mg/mL, 0.10 mg/mL, 0.16 mg/mL;
a concentration of pachymic acid is 0.01-0.20 mg/mL, for example, the concentration can be 0.01 mg/mL, 0.03 mg/mL, 0.10 mg/mL, 0.20 mg/mL.

Furthermore, detecting the test solution and the mixed reference solution by HPLC at 203 nm, including the following steps:
determining test conditions of HPLC and setting operating parameters of gradient elution;
injecting the test solution or the reference solution into a sample valve;
testing the test solution or reference solution according to the operating parameter program.

Furthermore, the test conditions of HPLC include:
a mobile phase is acetonitrile-phosphoric acid solution, where Phase A is acetonitrile and Phase B is phosphoric acid solution; a volume percentage concentration of the phosphoric acid solution is 0.09-0.11%, for example, 0.09%, 0.10%, 0.11%; a flow rate of the mobile phase is 0.95-1.05 mL/min, for example, 0.95 mL/min, 1.0 mL/min, 1.05 mL/min.

Furthermore, the test conditions of HPLC include:
a detector is a DAD detector, and a detection wavelength is 203 nm;
a chromatographic column is C18 chromatographic column, and a column temperature is 33-37° C., for example, 33° C., 35° C., 36° C. and 37° C.;
an injection volume is 10-20 μL, for example, 10 μL, 12 μL, 15 μL, 20 μL; an analysis time is 90 min, and gradient elution is adopted.

Furthermore, a filler in the chromatographic column is octadecylsilane bonded silica gel.

Furthermore, the operating parameter of gradient elution is as follows:
0-3 min, a volume percentage of Phase B is 85-84%, and a rest is Phase A;
3-5 min, the volume percentage of the Phase B is 84-78%, and the rest is the Phase A;
5-15 min, the volume percentage of the Phase B is 78%, and the rest is the Phase A;
15-18 min, the volume percentage of the Phase B is 78-75%, and the rest is the Phase A;
18-23 min, the volume percentage of the Phase B is 75-73%, and the rest is the Phase A;
23-27 min, the volume percentage of the Phase B is 73-67%, and the rest is the Phase A;
27-40 min, the volume percentage of the Phase B is 67-60%, and the rest is the Phase A;
40-45 min, the volume percentage of the Phase B is 60%, and the rest is the Phase A;
45-54 min, the volume percentage of the Phase B is 60-52%, and the rest is the Phase A;
54-59 min, the volume percentage of the Phase B is 52%, and the rest is the Phase A;
59-65 min, the volume percentage of the Phase B is 52-20%, and the rest is the Phase A;
65-90 min, the volume percentage of the Phase B is 20%, and the rest is the Phase A.

The beneficial effects of the invention are as follows:

The chromatographic conditions and elution gradients explored in this invention can quickly and comprehensively determine the contents of six characteristic components of ASDZW, polygalaxanthone III (representing ketones in polygala tenuifolia), 3,6′-disinapoyl sucrose (representing oligosaccharides in polygala tenuifolia), ginseng saponin Rb1 (representing ginseng saponins in panax ginseng), β-asarone (representing volatile oil in acori tatarinowii rhizoma), dehydrotumulosic acid and pachymic acid (including triterpenes of poria and poria with hostwood). The whole detection process is single wavelength detection, with high detection efficiency and short analysis time; the detection limits of the six components are lower than 0.008 μg/mL, indicating high sensitivity; the blank medium has no interference peak, indicating good specificity; the linear relationship r2 of the six characteristic components is greater than 0.999, and the results are accurate; the RSD of the peak area of the six characteristic components in the methodological investigation is less than 2%, indicating good reproducibility.

The detection method of multi-characteristic components related to the efficacy of ASDZW constructed by the invention can accurately determine the content of characteristic components, comprehensively reflect the efficacy of the compound, and provide a basis for the quality control and evaluation of ASDZW. Meanwhile, it provides a reference for the establishment of content determination methods in traditional Chinese medicine compound preparations containing polygala tenuifolia, ginseng, acori tatarinowii rhizoma, and poria, and provides quality control standard support for the development of new products.

Other features and advantages of the invention will be described in the subsequent specification, and partially become apparent from the specification, or be understood through the embodiment of the invention, the purpose and other advantages of the invention can be realized and obtained by the structure pointed out in the specification, the claim and the accompanying drawings.

### BRIEF DESCRIPTION OF THE DRAWINGS

In order to more clearly explain the technical scheme in the embodiment or the existing technology of the invention, the following will briefly introduce the drawings that need to be used in the embodiment or the existing technology description. Obviously, the drawings in the following description are some embodiments of the invention, for ordinary technicians in this field, they can also obtain other drawings based on these drawings without paying for creative labor.

FIG. 1 is an HPLC chromatogram of the mixed reference solution of the Anshen Dingzhi Wan (ASDZW) at the wavelength of 203 nm by the detection method proposed in the embodiment of the invention;

FIG. 2 is an HPLC chromatogram of the test solution of ASDZW at the wavelength of 203 nm by the detection method proposed in the embodiment of the invention;

FIG. 3 is an HPLC chromatogram of the blank medium at the wavelength of 203 nm of the detection method proposed in the embodiment of the invention;

FIG. 4 is an HPLC chromatogram of the negative test solution without polygala tenuifolia at the wavelength of 203 nm by the detection method proposed in the embodiment of the invention;

FIG. 5 is an HPLC chromatogram of the negative test solution without ginseng deficiency at the wavelength of 203 nm by the detection method proposed in the embodiment of the invention.

FIG. 6 is an HPLC chromatogram of the negative test solution without acori tatarinowii rhizoma at the wavelength of 203 nm by the detection method proposed in the embodiment of the invention.

FIG. 7 is an HPLC chromatogram of the negative test solution without poria at the wavelength of 203 nm by the detection method proposed in the embodiment of the invention.

FIG. 8 is an HPLC chromatogram of ASDZW at the wavelength of 203 nm by the detection method proposed in Comparison case 1.

### DETAILED DESCRIPTION OF THE EMBODIMENTS

In order to make the purpose, technical scheme, and advantages of the embodiment of the invention clearer, the following will clearly and completely explain the technical scheme of the embodiment of the invention in combination with the attached figure of the embodiment of the invention; obviously, the described embodiment is part of the embodiment of the invention, not all of the embodiment. Based on the embodiments in this invention, all other embodiments obtained by ordinary technicians in this field without making creative labor belong to the scope of protection of this invention.

### Embodiment 1

This embodiment proposes a method for detecting the content of Anshen Dingzhi Wan (ASDZW), including the following processes:
S1: Preparing the test solution of ASDZW;
a total of 5 g of the ASDZW reference sample is accurately weighed and placed in a conical flask with a stopper, 100 mL of water-saturated n-butanol solution is added accurately and sealed for 30 min; then ultrasonic extraction is performed for 30 min, and filtered; 25 mL of the filtrate is placed in an evaporation dish to dry, the residue is dissolved in methanol and transferred to a 5 mL volumetric flask and diluted to the scale; the mixture is shaken well and filtered through a 0.22 μm microporous filter membrane to obtain the continued filtrate, that is the test solution of ASDZW.
S2: Preparation of mixed reference solution of ASDZW;
the reference substances of polygalaxanthone III, 3,6′-disinapoyl sucrose, ginseng saponin Rb1, β-asarone, dehydrotumulosic acid, and pachymic acid are accurately weighed, dissolved and diluted with methanol solution to prepare mixed reference solution with different concentrations. In the reference solution, the concentration of polygalaxanthone III is 0.07 mg/mL, the concentration of 3,6′-disinapoyl sucrose is 0.25 mg/mL, the concentration of ginseng saponin Rb1 is 0.10 mg/mL, the concentration of β-asarone is 0.10 mg/mL, the concentration of dehydrotumulosic acid is 0.03 mg/mL, and the concentration of pachymic acid is 0.04 mg/mL.
S3: The test solution and mixed reference solution are determined by high performance liquid chromatography (HPLC);
the test conditions of HPLC are as follows: the mobile phase is acetonitrile-0.1% phosphoric acid solution, where Phase A is acetonitrile and Phase B is 0.1% phosphoric acid solution; the flow rate is 1.0 mL/min. The detector is a DAD detector; the detection wavelength is 203 nm, the chromatographic column is C18 chromatographic column; the filler in the chromatographic column is octadecylsilane bonded silica gel; the column temperature is 35° C., the injection volume is 10 μL; the analysis time is 90 min; gradient elution is performed.

The specific procedure of gradient elution is as follows:
0-3 min, the volume percentage of Phase B is 85-84%, and the rest is Phase A;
3-5 min, the volume percentage of the Phase B is 84-78%, and the rest is the Phase A;
5-15 min, the volume percentage of the Phase B is 78%, and the rest is the Phase A;
15-18 min, the volume percentage of the Phase B is 78-75%, and the rest is the Phase A;
18-23 min, the volume percentage of the Phase B is 75-73%, and the rest is the Phase A;
23-27 min, the volume percentage of the Phase B is 73-67%, and the rest is the Phase A;
27-40 min, the volume percentage of the Phase B is 67-60%, and the rest is the Phase A;
40-45 min, the volume percentage of the Phase B is 60%, the rest is the Phase A;
45-54 min, the volume percentage of the Phase B is 60-52%, and the rest is the Phase A;
54-59 min, the volume percentage of the Phase B is 52%, and the rest is the Phase A.
59-65 min, the volume percentage of the Phase B is 52-20%, and the rest is the Phase A.
65-90 min, the volume percentage of the Phase B is 20%, and the rest is the Phase A.

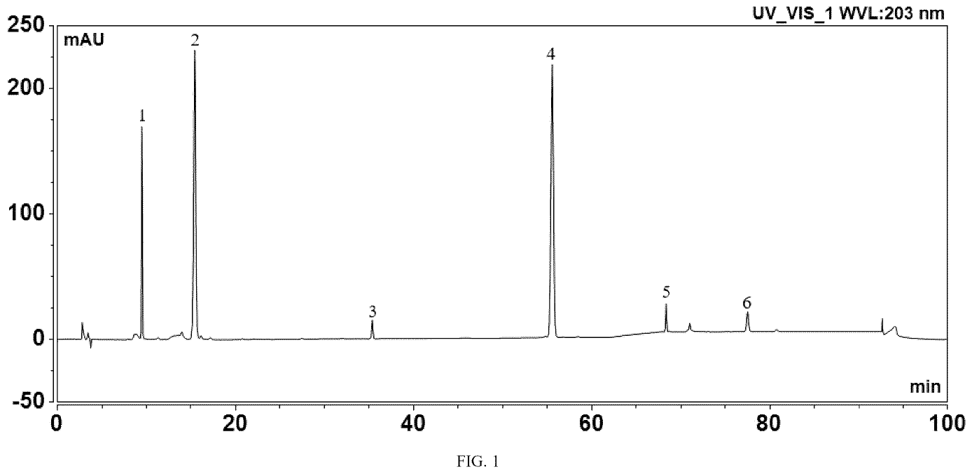

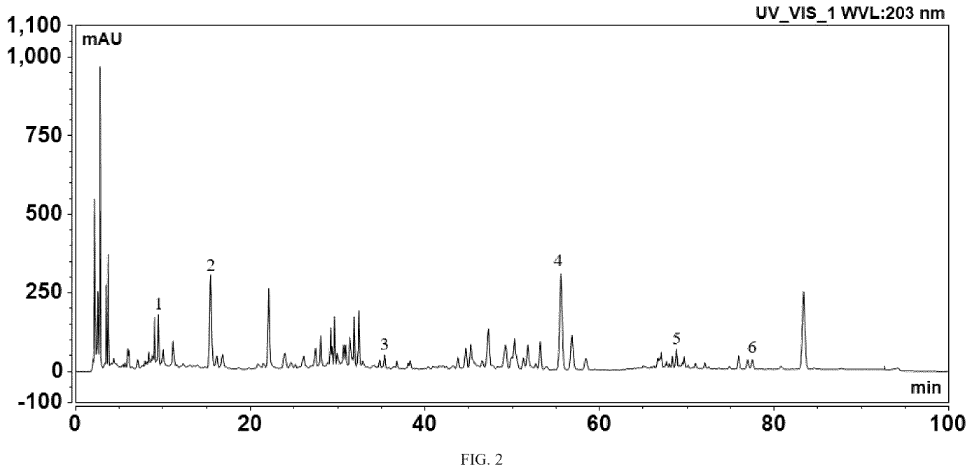

The liquid chromatography of the mixed reference solution and the test solution of ASDZW in this embodiment are shown in FIG. 1 and FIG. 2 respectively. In FIG. 1, Peak 1 is the characteristic peak of the polygalaxanthone III, Peak 2 is the characteristic peak of the 3,6′-disinapoyl sucrose, Peak 3 is the characteristic peak of the ginseng saponin Rb1, Peak 4 is the characteristic peak of the β-asarone, Peak 5 is the characteristic peak of the dehydrotumulosic acid, and Peak 6 is the characteristic peak of the pachymic acid.

S4: The liquid chromatogram of the test solution and the liquid chromatogram of the mixed reference solution are compared to determine the characteristic peaks in ASDZW and calculate the content of each component according to the comparison results.

Comparing the peak time of each characteristic peak in FIG. 1 and FIG. 2, it can be determined that Peak 1 in FIG. 2 is the characteristic peak of the polygalaxanthone III, Peak 2 is the 3,6′-disinapoyl sucrose, Peak 3 is the characteristic peak of the ginseng saponin Rb1, Peak 4 is the characteristic peak of the β-asarone, Peak 5 is the characteristic peak of the dehydrotumulosic acid, and Peak 6 is the characteristic peak of the pachymic acid.

Specifically, according to the chromatographic conditions in S3, the mixed reference solution and the sixteen batches of ASDZW reference samples are were injected and measured their average peak area, then the content of each component in the sample was calculated by external standard one-point method; the content determination results are shown in Table 1:

TABLE 1
Content determination results of six components in sixteen batches of ASDZW reference samples

| | | 3,6′- | | | | |
|---|---|---|---|---|---|---|
| | Polygalaxanthone | disinapoyl | Ginseng | β- | Dehydrotumulosic | Pachymic |
| Batch | III(mg/g) | sucrose(mg/g) | saponinRb1(mg/g) | asarone(mg/g) | acid(mg/g) | acid(mg/g) |
| ASDZW202401 | 0.08 | 0.24 | 0.15 | 0.19 | 0.04 | 0.05 |
| ASDZW202402 | 0.07 | 0.25 | 0.15 | 0.20 | 0.04 | 0.05 |
| ASDZW202403 | 0.09 | 0.25 | 0.14 | 0.21 | 0.03 | 0.04 |
| ASDZW202404 | 0.07 | 0.25 | 0.13 | 0.21 | 0.04 | 0.03 |
| ASDZW202405 | 0.08 | 0.24 | 0.14 | 0.21 | 0.05 | 0.03 |
| ASDZW202406 | 0.08 | 0.25 | 0.14 | 0.23 | 0.04 | 0.05 |
| ASDZW202407 | 0.09 | 0.26 | 0.14 | 0.19 | 0.03 | 0.04 |
| ASDZW202408 | 0.06 | 0.24 | 0.13 | 0.18 | 0.02 | 0.06 |
| ASDZW202409 | 0.08 | 0.24 | 0.15 | 0.19 | 0.04 | 0.04 |
| ASDZW202410 | 0.06 | 0.25 | 0.15 | 0.18 | 0.03 | 0.05 |
| ASDZW202411 | 0.06 | 0.25 | 0.16 | 0.20 | 0.03 | 0.03 |
| ASDZW202412 | 0.08 | 0.25 | 0.16 | 0.20 | 0.04 | 0.04 |
| ASDZW202413 | 0.09 | 0.25 | 0.15 | 0.22 | 0.03 | 0.05 |
| ASDZW202414 | 0.09 | 0.23 | 0.15 | 0.21 | 0.02 | 0.05 |
| ASDZW202415 | 0.08 | 0.25 | 0.15 | 0.19 | 0.04 | 0.05 |
| ASDZW202416 | 0.08 | 0.23 | 0.15 | 0.19 | 0.03 | 0.04 |

### Embodiment 2

Methodological verification of the content detection method of polygalaxanthone III, 3,6′-disinapoyl sucrose, ginseng saponin Rb1, β-asarone, dehydrotumulosic acid and pachymic acid in ASDZW proposed in Embodiment 1, including the specificity of the detection method, the linear relationship of the detection method, stability, precision, accuracy and durability, the performance indicators are as follows.
(1) Specificity detection of detection methods;
10 μL of the blank solvent methanol, 10 μL of mixed reference solution, 10 μL of sample-deficient negative reference solution, and 10 μL of test solution are accurately extracted respectively and injected into the liquid chromatograph; the detection is performed according to the determination conditions in S3 of Embodiment 1, and the liquid chromatograms are recorded and shown in FIG. 1-FIG. 7.

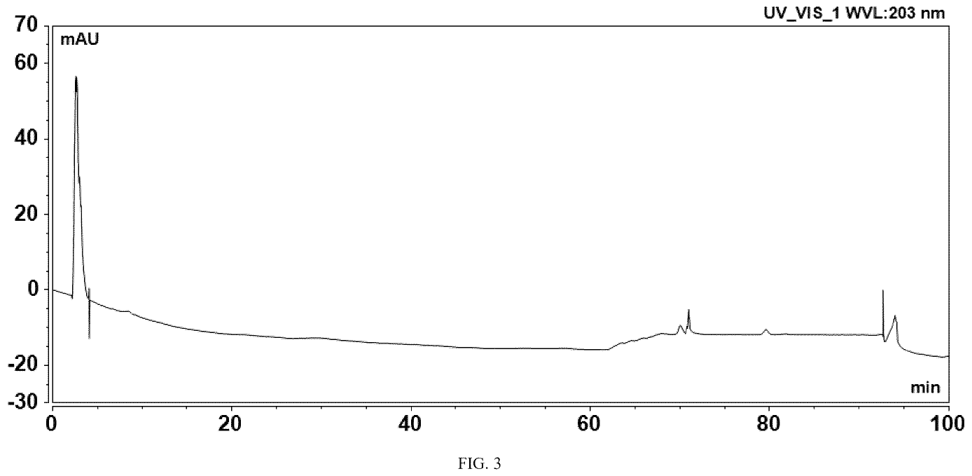

Determination of blank solvent methanol: According to the determination conditions of S3 in Embodiment 1, the blank solvent methanol is detected, the obtained liquid chromatogram is shown in FIG. 3, and the blank medium has no interference peak, meaning good specificity.

Determination of mixed reference solution: The mixed reference solution is prepared in S2 of Embodiment 1, and the obtained liquid chromatogram is shown in FIG. 1. Among them, Peak 1 is the characteristic peak of the polygalaxanthone III, Peak 2 is the characteristic peak of the 3,6′-disinapoyl sucrose, Peak 3 is the characteristic peak of the ginseng saponin Rb1, Peak 4 is the characteristic peak of the β-asarone, Peak 5 is the characteristic peak of the dehydrotumulosic acid, and Peak 6 is the characteristic peak of the pachymic acid; it can be seen that the characteristic peaks of each reference solution are obvious and do not interfere with each other.

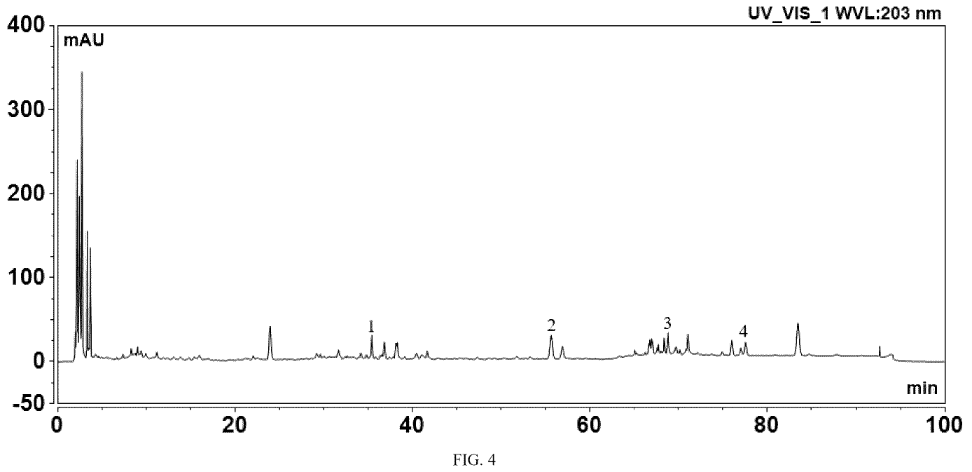

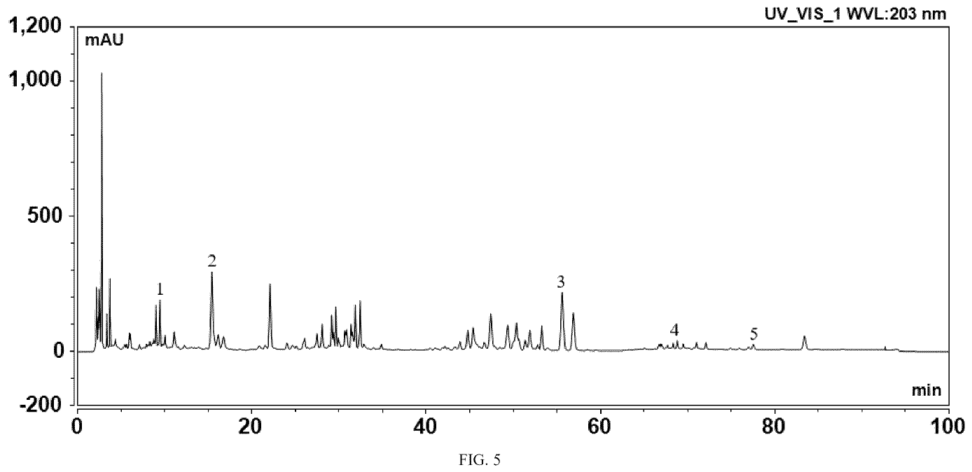

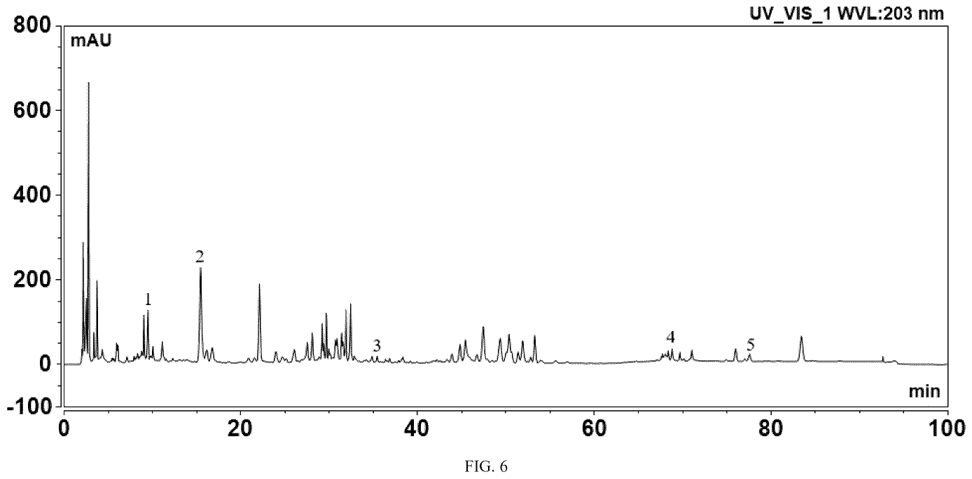

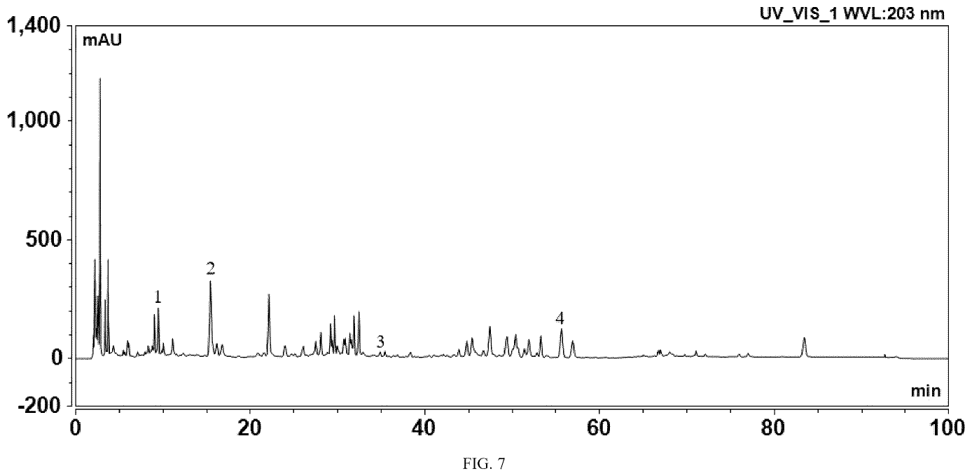

Determination of sample-deficient negative reference solutions: Any one of the herbs in Anshen Dingzhi Wan sample is removed and prepared according to S1 in Embodiment 1 to obtain the negative test solution without polygala tenuifolia, the negative test solution without ginseng, the negative test solution without acori tatarinowii rhizoma, and the negative test solution without poria. The liquid chromatograms of the sample-deficient negative test solutions are shown in FIG. 4-FIG. 7. FIG. 4 is the liquid chromatogram of the negative test solution without polygala tenuifolia, where Peak 1 is the characteristic peak of the ginseng saponin Rb1; Peak 2 is the characteristic peak of the β-asarone; Peak 3 is the characteristic peak of the dehydrotumulosic acid, and Peak 4 is the characteristic peak of the pachymic acid. FIG. 5 is the liquid chromatogram of the negative test solution without ginseng, where Peak 1 is the characteristic peak of the polygalaxanthone III, Peak 2 is the characteristic peak of the 3,6′-disinapoyl sucrose, Peak 3 is the characteristic peak of the β-asarone, Peak 4 is the characteristic peak of the dehydrotumulosic acid, and Peak 5 is the characteristic peak of the pachymic acid. FIG. 6 is the liquid chromatogram of the negative test solution without acori tatarinowii rhizoma, where Peak 1 is the characteristic peak of the polygalaxanthone III, Peak 2 is the characteristic peak of the 3,6′-disinapoyl sucrose, Peak 3 is the characteristic peak of the ginseng saponin Rb1, Peak 4 is the characteristic peak of the dehydrotumulosic acid, and Peak 5 is the characteristic peak of the pachymic acid. FIG. 7 is the liquid chromatogram of the negative test solution without poria, where Peak 1 is the characteristic peak of the polygalaxanthone III, Peak 2 is the characteristic peak of the 3,6′-disinapoyl sucrose, Peak 3 is the characteristic peak of the ginseng saponin Rb1, and Peak 4 is the characteristic peak of the β-asarone.

Determination of the test solution: The test solution is prepared in S1 in Embodiment 1, and the liquid chromatogram is shown in FIG. 2, by comparing FIG. 1 and FIG. 4-FIG. 7, six characteristic peaks in the HPLC chromatogram of ASDZW can be determined, where Peak 1 is the characteristic peak of the polygalaxanthone III, Peak 2 is the characteristic peak of the 3,6′-disinapoyl sucrose, Peak 3 is the characteristic peak of the ginseng saponin Rb1, Peak 4 is the characteristic peak of the β-asarone, Peak 5 is the characteristic peak of the dehydrotumulosic acid, and Peak 6 is the characteristic peak of the pachymic acid; from FIG. 1-FIG. 7, the retention times of the characteristic peaks are the polygalaxanthone III characteristic peak (9.463-9.467), the 3,6′-disinapoyl sucrose characteristic peak (15.387-15.423), the ginseng saponin Rb1 characteristic peak (35.413-35.433), the β-asarone characteristic peak (55.617-55.623), the dehydrotumulosic acid characteristic peak (68.343-68.347), the pachymic acid characteristic peak (77.533-77.547); the results show that the negative test does not interfere with the determination of the content of the product, and the method has good specificity.
(2) Detection of the linear relationship of the detection method;
appropriate amounts of the reference substances of polygalaxanthone III, 3,6′-disinapoyl sucrose, ginseng saponin Rb1, β-asarone, dehydrotumulosic acid, and pachymic acid are accurately weighed, and a series of reference solutions with different concentrations are prepared by adding methanol solution according to S2 in Embodiment 1. According to the chromatographic conditions of S3 in Embodiment 1, 10 μL of mixed reference solution is accurately absorbed and injected into the HPLC, the standard curve is drawn with the reference concentration as the ordinate and the peak area of each characteristic component as the abscissa, the specific data of the standard curve are shown in Table 2; meanwhile, the standard regression equation, correlation coefficient and linear range of polygalaxanthone III, 3,6′-disinapoyl sucrose, ginseng saponin Rb1, β-asarone, dehydrotumulosic acid and pachymic acid are obtained by calculation, and the specific results are shown in Table 2.

TABLE 2
Standard curve data of six characteristic components

| | | Correlation | |
|---|---|---|---|
| | | coefficient | Linear |
| Component | Linear equation | r² | range(mg/mL) |
| Polygalaxanthone | y = 264.29x + 1.2017 | 0.9993 | 0.01-0.32 |
| III | | | |
| 3,6′-disinapoyl | y = 228.06x + 3.5805 | 0.9992 | 0.03125-1.00 |
| sucrose | | | |
| Ginseng | y = 24.709x + 0.1679 | 0.9997 | 0.01875-0.60 |
| saponinRb1 | | | |
| β-asarone | y = 778.89x + 7.8276 | 0.9996 | 0.02375-0.76 |
| Dehydrotumu- | y = 74.964x + 0.1328 | 0.9997 | 0.005-0.16 |
| losic acid | | | |
| Pachymic acid | y = 106.7x + 0.2244 | 0.9998 | 0.00625-0.2 |

According to Table 2, it can be seen that the six characteristic components have a good linear relationship in their respective injection concentration ranges, indicating that the method has a wide linear range and high accuracy.
(3) Stability detection of the detection method;
the samples of ASDZW with a batch number of ASDZW202405 are taken to prepare the test solution according to S1 in Embodiment 1 and analyzed according to the chromatographic conditions of S3 in Embodiment 1 at 0 h, 2 h, 4 h, 6 h, 8 h, 12 h and 24 h, respectively. The specific results are shown in Table 3.

TABLE 3
Stability test results

| | | | 3,6′- | | | | | | | | | |
|---|---|---|---|---|---|---|---|---|---|---|---|---|
| | Polygalaxanthone | | disinapoyl | | Ginseng | | | | Dehydrotumulosic | | Pachymic | |
| | III | | sucrose | | saponinRb1 | | β-asarone | | acid | | acid | |
| | Peak area | | Peak area | | Peak area | | Peak area | | Peak area | | Peak area | |
| Time | RSD | | RSD | | RSD | | RSD | | RSD | | RSD | |
| 0 h | 16.8433 | 0.69% | 63.4213 | 0.40% | 4.2737 | 0.57% | 81.5747 | 1.82% | 2.3496 | 1.74% | 5.0182 | 1.27% |
| 2 h | 16.8483 | | 63.4218 | | 4.3048 | | 81.6057 | | 2.3760 | | 5.1014 | |
| 4 h | 16.8592 | | 63.4262 | | 4.2196 | | 81.6049 | | 2.3562 | | 5.0866 | |
| 6 h | 16.8569 | | 63.4273 | | 4.4337 | | 81.5956 | | 2.3519 | | 5.1854 | |
| 8 h | 16.8519 | | 63.4263 | | 4.4701 | | 81.6095 | | 2.3426 | | 5.1147 | |
| 12 h | 16.8645 | | 63.4435 | | 4.5378 | | 81.5302 | | 2.3449 | | 5.0515 | |
| 24 h | 16.8824 | | 63.4583 | | 4.0197 | | 81.6082 | | 2.4091 | | 5.1952 | |

The results show that the RSDs of the chromatographic peak areas of polygalaxanthone III, 3,6′-disinapoyl sucrose, ginseng saponin Rb1, β-asarone, dehydrotumulosic acid, and pachymic acid are less than 2% within 24 h, indicating that the test solution had no effect on the results within 24 h, and the stability of the detection method is good.
(4) The precision test of the detection method;
4.1) intermediate precision detection;
the same batch of ASDZW samples with a batch number of ASDZW202405 are taken by different experimenters, and three test solutions are prepared using S1 in Embodiment 1; according to the chromatographic conditions of S3 in Embodiment 1, the samples are injected and analyzed on the same HPLC, and the peak area results are shown in Table 4.

TABLE 4
Intermediate precision measurement results

| | Polygalaxanthone | | 3,6′- | | Ginseng | | | Dehydrotumulosic | | | | |
|---|---|---|---|---|---|---|---|---|---|---|---|---|
| | III | | disinapoylsucrose | | saponin | β-asarone | | acid | | Pachymic acid | | |
| | Peak | | Peak | | Rb1 | | Peak | | Peak | | Peak | |
| No. | area | RSD | area | RSD | Peak area | RSD | area | RSD | area | RSD | area | RSD |
| 1 | 26.5160 | 1.03% | 93.1625 | 0.93% | 5.0399 | 1.04% | 111.4865 | 0.75% | 4.5936 | 0.58% | 7.8965 | 0.71% |
| 2 | 26.3434 | | 91.3097 | | 5.0024 | | 112.5980 | | 4.5515 | | 7.7377 | |
| 3 | 26.4106 | | 92.0087 | | 5.0574 | | 111.7970 | | 4.5757 | | 7.7787 | |
| 4 | 26.6274 | | 91.9534 | | 5.1067 | | 111.5824 | | 4.5789 | | 7.7330 | |
| 5 | 27.1774 | | 93.8696 | | 5.1505 | | 112.6218 | | 4.6352 | | 7.8383 | |
| 6 | 26.9425 | | 92.9660 | | 5.1472 | | 113.6934 | | 4.6171 | | 7.8032 | |

The results show that the RSD of the peak area of the six characteristic components in ASDZW measured by different experimenters using the same instrument is less than 2%, indicating good precision of the instrument.
4.2) repeatability test;
the samples of ASDZW with a batch number of ASDZW202405 are prepared in parallel according to S1 in Embodiment 1 to obtain 6 test solutions, and each sample solution is measured twice according to the chromatographic conditions of S3 in Embodiment 1; the specific results of the peak area repeatability are shown in Table 5.

TABLE 5
Repeatability test results

| | | 3,6′- | Ginseng | | | |
|---|---|---|---|---|---|---|
| | Polygalaxanthone | disinapoyl | saponin | | Dehydrotumulosic | Pachymic |
| Sample | III | sucrose | Rb1 | β-asarone | acid | acid |
| Sample | 16.6686 | 61.3388 | 4.0541 | 80.3428 | 2.7767 | 4.9431 |
| 1 | | | | | | |
| Sample | 16.3119 | 60.3532 | 3.9610 | 79.2800 | 2.5267 | 5.0122 |
| 2 | | | | | | |
| Sample | 16.8776 | 61.9086 | 4.0632 | 81.3277 | 2.6750 | 5.0008 |
| 3 | | | | | | |
| Sample | 16.6610 | 61.3024 | 4.0583 | 81.0489 | 2.5994 | 5.0816 |
| 4 | | | | | | |
| Sample | 16.8960 | 62.4024 | 4.1205 | 80.4102 | 2.1336 | 5.5504 |
| 5 | | | | | | |
| Sample | 17.1678 | 62.5827 | 4.1714 | 80.9873 | 2.6568 | 5.1399 |
| 6 | | | | | | |
| Mean | 16.9020 | 62.0871 | 4.1054 | 81.2295 | 2.5990 | 5.0550 |
| value | | | | | | |
| SD | 0.0000041 | 0.0000086 | 0.0000085 | 0.0000021 | 0.0000026 | 0.0000030 |
| RSD | 1.07% | 0.71% | 1.03% | 0.47% | 1.46% | 1.25% |

The results show that the RSD of the peak area of the six characteristic components is less than 2%, which met the requirements of the 2020 edition of Chinese Pharmacopoeia, indicating good repeatability of this method.
(5) Detection of the sample recovery rate of detection method;
six samples of ASDZW (batch number ASDZW202405) with known concentrations (2.5 g) are accurately weighed, and the mixed reference solution of six characteristic components is added at three levels of 50%, 100%, and 150% of the content, respectively, and each level is parallel to 3 copies. The test solution is prepared according to S1 in Embodiment 1, and the sample is injected and analyzed according to the chromatographic conditions of S3 in Embodiment 1. The results are shown in Table 6.

TABLE 6
Determination results of sample recovery rate

| | | | Standard | | Average | |
|---|---|---|---|---|---|---|
| | | Sample | addition | Sample | sample | |
| Detected | Sampling | medium | amount | recovery | recovery | |
| component | volume (g) | volume(mg) | (mg) | rate(%) | rate(%) | RSD(%) |
| Polygalaxanthone | 0.2154 | 0.4154 | 0.21 | 95.2469 | 95.130 | 1.85 |
| III | 0.2199 | 0.4202 | 0.21 | 95.4047 | | |
| | 0.2160 | 0.4131 | 0.21 | 93.8659 | | |
| | 0.2192 | 0.4144 | 0.21 | 92.9321 | | |
| | 0.2130 | 0.4190 | 0.21 | 98.1228 | | |
| | 0.2213 | 0.4212 | 0.21 | 95.2074 | | |
| 3,6′- | 0.8096 | 1.5578 | 0.81 | 92.3753 | 93.059 | 1.206 |
| disinapoyl | 0.8492 | 1.6017 | 0.81 | 92.9073 | | |
| sucrose | 0.8042 | 1.5486 | 0.81 | 91.8983 | | |
| | 0.8052 | 1.5528 | 0.81 | 92.2920 | | |
| | 0.8135 | 1.5797 | 0.81 | 94.5964 | | |
| | 0.8314 | 1.5951 | 0.81 | 94.2845 | | |
| Ginseng | 1.1928 | 2.3366 | 1.20 | 95.3118 | 94.236 | 1.547 |
| saponinRb1 | 1.2033 | 2.3068 | 1.20 | 91.9597 | | |
| | 1.1935 | 2.3302 | 1.20 | 94.7257 | | |
| | 1.2167 | 2.3496 | 1.20 | 94.4034 | | |
| | 1.1654 | 2.3162 | 1.20 | 95.8978 | | |
| | 1.2312 | 2.3487 | 1.20 | 93.1200 | | |
| β-asarone | 1.2678 | 2.4877 | 1.24 | 98.3785 | 99.466 | 0.767 |
| | 1.2357 | 2.4667 | 1.24 | 99.2753 | | |
| | 1.2229 | 2.4590 | 1.24 | 99.6836 | | |
| | 1.2432 | 2.4797 | 1.24 | 99.7201 | | |
| | 1.2475 | 2.4760 | 1.24 | 99.0785 | | |
| | 1.2560 | 2.5042 | 1.24 | 100.6606 | | |
| Dehydrotumulosic | 1.3726 | 2.6304 | 1.30 | 96.7559 | 94.4453 | 1.58 |
| acid | 1.4063 | 2.6124 | 1.30 | 92.7758 | | |
| | 1.3891 | 2.6170 | 1.30 | 94.4500 | | |
| | 1.4106 | 2.6321 | 1.30 | 93.9654 | | |
| | 1.3729 | 2.6149 | 1.30 | 95.5372 | | |
| | 1.4211 | 2.6326 | 1.30 | 93.1878 | | |
| Pachymic | 0.2076 | 0.4185 | 0.21 | 100.4523 | 99.437 | 0.828 |
| acid | 0.2070 | 0.4152 | 0.21 | 99.1205 | | |
| | 0.2098 | 0.4166 | 0.21 | 98.4842 | | |
| | 0.2097 | 0.4203 | 0.21 | 100.2954 | | |
| | 0.2129 | 0.4220 | 0.21 | 99.5976 | | |
| | 0.2200 | 0.4272 | 0.21 | 98.6730 | | |

It can be seen from Table 6 that the recovery rates of the six characteristic components are all in line with the recovery limit specified in the 2020 edition of Chinese Pharmacopoeia (Volume IV), indicating that the accuracy of the determination method is good.
(6) Durability test of the detection method;
ASDZW samples with a batch number ASDZW202405 are taken to prepare the test solution in S1 in Embodiment 1. According to the chromatographic conditions of S3 in Embodiment 1, the chromatographic solution was determined under different types of chromatographic columns, different column temperatures, flow rates, different mobile phase ratios, and different extraction time, and the peak area results are shown in Table 7-11.

TABLE 7
Durability (chromatographic column type)

| | | 3,6′- | | | | |
|---|---|---|---|---|---|---|
| | Polygalaxanthone | disinapoyl | Ginseng | | Dehydrotumulosic | Pachymic |
| Type | III | sucrose | saponinRb1 | β-asarone | acid | acid |
| ThermoFisher | 2.3139 | 7.4827 | 1.7818 | 1.5510 | 50.7600 | 5.1617 |
| C18 | | | | | | |
| Pharomen H23- | 2.2648 | 7.4875 | 1.7874 | 1.5465 | 50.7113 | 5.1799 |
| 122401 | | | | | | |
| Agilent | 2.2880 | 7.6459 | 1.7782 | 1.5763 | 50.6370 | 5.0832 |
| USNH030933 | | | | | | |
| Mean value | 2.2889 | 7.5387 | 1.7825 | 1.5579 | 50.7028 | 5.1416 |
| SD | 0.0246 | 0.0929 | 0.0046 | 0.0161 | 0.0619 | 0.0514 |
| RSD(%) | 1.07 | 1.23 | 0.26 | 1.03 | 0.12 | 1.00 |

TABLE 8
Durability (column temperature)

| | | 3,6′- | Ginseng | | | |
|---|---|---|---|---|---|---|
| Column | Polygalaxanthone | disinapoyl | saponin | β- | Dehydrotumulosic | Pachymic |
| temperature | III | sucrose | Rb1 | asarone | acid | acid |
| Column | 2.3609 | 7.5736 | 1.6999 | 1.5110 | 51.2188 | 4.5893 |
| temperature 29° C. | | | | | | |
| Column | 2.3669 | 7.5758 | 1.6860 | 1.5085 | 51.2734 | 4.5722 |
| temperature 30° C. | | | | | | |
| Column | 2.3639 | 7.5726 | 1.6967 | 1.5151 | 51.1411 | 4.5835 |
| temperature 31° C. | | | | | | |
| Mean value | 2.3639 | 7.5740 | 1.6942 | 1.5115 | 51.2111 | 4.5817 |
| SD | 0.0030 | 0.0016 | 0.0073 | 0.0033 | 0.0066 | 0.0087 |
| RSD(%) | 0.13 | 0.02 | 0.43 | 0.22 | 0.13 | 0.19 |

TABLE 9
Durability (flow rate)

| | | 3,6′- | | | | |
|---|---|---|---|---|---|---|
| | Polygalaxanthone | disinapoyl | Ginseng | β- | Dehydrotumulosic | Pachymic |
| Flow rate | III | sucrose | saponinRb1 | asarone | acid | acid |
| 0.99 mL/min | 0.1761 | 0.6233 | 1.1772 | 1.4208 | 1.2254 | 0.2282 |
| 1.0 mL/min | 0.1769 | 0.6327 | 1.1782 | 1.4229 | 1.2287 | 0.2305 |
| 1.01 mL/min | 0.1738 | 0.6218 | 1.1745 | 1.4255 | 1.2164 | 0.2289 |
| Mean value | 0.1756 | 0.6260 | 1.1766 | 1.4230 | 1.2235 | 0.2292 |
| SD | 0.0013 | 0.0048 | 0.0016 | 0.0019 | 0.0052 | 0.0010 |
| RSD(%) | 0.94 | 0.95 | 0.17 | 0.17 | 0.53 | 0.52 |

TABLE 10
Durability (acid water mobile phase ratio)

| | | 3,6′- | | | | |
|---|---|---|---|---|---|---|
| | Polygalaxanthone | disinapoyl | Ginseng | β- | Dehydrotumulosic | Pachymic |
| Acid water mobile phase ratio | III | sucrose | saponinRb1 | asarone | acid | acid |
| Mobile phase 0.01% phosphoric | 0.2181 | 0.8383 | 1.2442 | 1.4068 | 1.3677 | 0.2092 |
| acid solution | | | | | | |
| Mobile phase 1.00% phosphoric | 0.2158 | 0.8319 | 1.2345 | 1.3965 | 1.3622 | 0.2099 |
| acid solution | | | | | | |
| Mobile phase 0.09% phosphoric | 0.2165 | 0.8368 | 1.2227 | 1.3937 | 1.3719 | 0.2102 |
| acid solution | | | | | | |
| Mean value | 0.2168 | 0.8357 | 1.2338 | 1.3990 | 1.3673 | 0.2098 |
| SD | 0.0010 | 0.0027 | 0.0088 | 0.0056 | 0.0040 | 0.0004 |
| RSD(%) | 0.55 | 0.41 | 0.88 | 0.50 | 0.36 | 0.26 |

TABLE 11
Durability (sample extraction time)

| Sample | | 3,6′- | | | | |
|---|---|---|---|---|---|---|
| extraction | Polygalaxanthone | disinapoyl | Ginseng | β- | Dehydrotumulosic | Pachymic |
| time | III | sucrose | saponinRb1 | asarone | acid | acid |
| 25 min | 0.1770 | 0.6362 | 1.1504 | 1.4732 | 1.2686 | 0.2161 |
| 30 min | 0.1773 | 0.6393 | 1.1492 | 1.4910 | 1.2561 | 0.2157 |
| 35 min | 0.1760 | 0.6298 | 1.1436 | 1.4865 | 1.2692 | 0.2168 |
| Mean | 1.1767 | 0.6350 | 1.1477 | 1.4836 | 1.2646 | 0.2162 |
| value | | | | | | |
| SD | 0.0006 | 0.0040 | 0.0030 | 0.0075 | 0.0060 | 0.0004 |
| RSD(%) | 0.40 | 0.77 | 0.32 | 0.63 | 0.59 | 0.25 |

The results show that after changing the corresponding conditions, the RSD of the peak area of the six characteristic components is less than 2%, indicating that the content determination method had good durability.

### Comparison Case 1

The test solution of ASDZW are determined according to the steps of embodiments 1-3, the difference between this comparison case and Embodiment 1 is that the data ratio of Phase A and Phase B in the elution procedure is as follows:
0-10 min, the volume percentage of Phase B is 95-85%, and the rest is Phase A;
10-20 min, the volume percentage of the Phase B is 85-84%, and the rest is the Phase A;
20-31 min, the volume percentage of the Phase B is 84-82%, and the rest is the Phase A;
31-33 min, the volume percentage of the Phase B is 82-77%, and the rest is the Phase A;
33-52 min, the volume percentage of the Phase B is 77-60%, and the rest is the Phase A;
52-70 min, the volume percentage of the Phase B is 60-48%, and the rest is the Phase A;
70-76 min, the volume percentage of the Phase B is 48-27%, and the rest is the Phase A;
76-93 min, the volume percentage of the Phase B is 27-20%, and the rest is the Phase A;
93-100 min, the volume percentage of the Phase B is 20-5%, and the rest is the Phase A;
100-102 min, the volume percentage of the Phase B is 5%, and the rest is the Phase A.

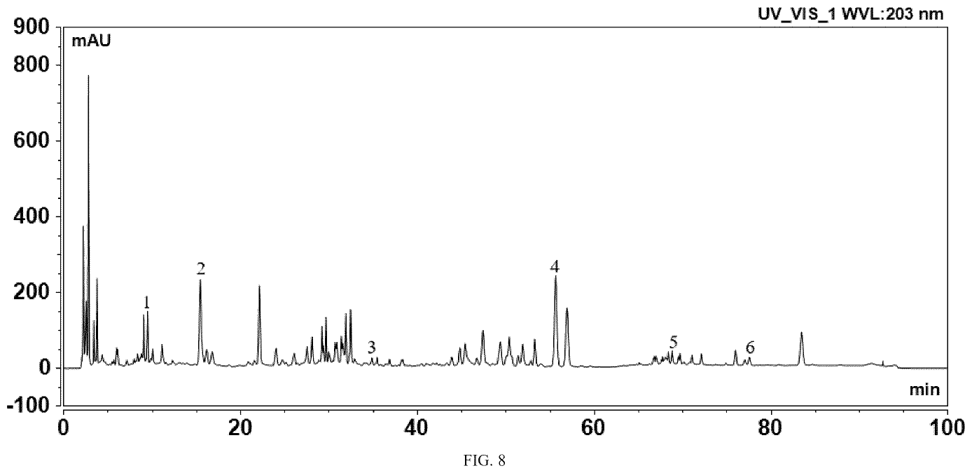

According to the above chromatographic conditions, the obtained HPLC is shown in FIG. 8. It can be seen that the detected characteristic peaks of Peak 1, Peak 5, and Peak 6 are not obvious and accompanied by the appearance of miscellaneous peaks, which interferes with the accuracy of the detection data.

The accuracy of the detection method of Comparison case 1 is verified by the sample recovery rate, as follows:

TABLE 12
Determination results of sample recovery rate

| | | | | | Average | |
|---|---|---|---|---|---|---|
| | | Solution | | Sample | sample | |
| Detected | Sampling | after | Standard | recovery | recovery | |
| component | volume(mg) | labeling(mg/g) | substance(mg/g) | rate(%) | rate(%) | RSD(%) |
| Polygalaxanthone | 0.2154 | 0.3954 | 0.21 | 85.7231 | 88.7808 | 5.405 |
| III | 0.2199 | 0.4002 | 0.21 | 85.8809 | | |
| | 0.2160 | 0.4031 | 0.21 | 89.1040 | | |
| | 0.2192 | 0.4044 | 0.21 | 88.1702 | | |
| | 0.2130 | 0.4190 | 0.21 | 98.1228 | | |
| | 0.2213 | 0.4012 | 0.21 | 85.6836 | | |
| 3,6′- | 0.8096 | 1.5778 | 0.81 | 94.8444 | 88.7380 | 4.248 |
| disinapoyl | 0.8492 | 1.5317 | 0.81 | 84.2653 | | |
| sucrose | 0.8042 | 1.5386 | 0.81 | 90.6637 | | |
| | 0.8052 | 1.5128 | 0.81 | 87.3538 | | |
| | 0.8135 | 1.5097 | 0.81 | 85.9544 | | |
| | 0.8314 | 1.5551 | 0.81 | 89.3463 | | |
| Ginseng | 1.1928 | 2.2566 | 1.20 | 88.6451 | 85.7642 | 3.169 |
| saponinRb1 | 1.2033 | 2.2068 | 1.20 | 83.6263 | | |
| | 1.1935 | 2.2402 | 1.20 | 87.2257 | | |
| | 1.2167 | 2.2496 | 1.20 | 86.0701 | | |
| | 1.1654 | 2.2162 | 1.20 | 87.5645 | | |
| | 1.2312 | 2.2087 | 1.20 | 81.4534 | | |
| β- | 1.2678 | 2.3477 | 1.24 | 87.0882 | 89.2511 | 3.068 |
| asarone | 1.2357 | 2.3067 | 1.24 | 86.3721 | | |
| | 1.2229 | 2.3590 | 1.24 | 91.6191 | | |
| | 1.2432 | 2.3697 | 1.24 | 90.8491 | | |
| | 1.2475 | 2.3260 | 1.24 | 86.9817 | | |
| | 1.2560 | 2.4042 | 1.24 | 92.5961 | | |
| Dehydrotumulosic | 1.3726 | 2.5804 | 1.30 | 92.9098 | 87.9069 | 3.208 |
| acid | 1.4063 | 2.5124 | 1.30 | 85.0835 | | |
| | 1.3891 | 2.5170 | 1.30 | 86.7577 | | |
| | 1.4106 | 2.5321 | 1.30 | 86.2731 | | |
| | 1.3729 | 2.5049 | 1.30 | 87.0756 | | |
| | 1.4211 | 2.5826 | 1.30 | 89.3417 | | |
| Pachymic | 0.2076 | 0.4085 | 0.21 | 95.6904 | 89.9134 | 4.032 |
| acid | 0.2070 | 0.3952 | 0.21 | 89.5966 | | |
| | 0.2098 | 0.3966 | 0.21 | 88.9604 | | |
| | 0.2097 | 0.4003 | 0.21 | 90.7716 | | |
| | 0.2129 | 0.4020 | 0.21 | 90.0738 | | |
| | 0.2200 | 0.3972 | 0.21 | 84.3873 | | |

The results show that compared with the detection data of Comparison case 1, the detection method of the invention has shorter analysis time; the detection limits of the six components are lower than 0.008 μg/mL, meaning high sensitivity; the blank medium has no interference peak with good specificity; the linear relationship r2 of the six characteristic components is greater than 0.999, indicating accurate results; the peak area RSD of the six characteristic components in the methodological investigation is less than 2%, indicating good reproducibility. The data in Comparison case 1 show that after changing the ratio of Phase A and Phase B in the elution procedure, the average recovery rate of the determination method is lower than that of Embodiment 1, and the RSD value of Comparison case 1 is greater than 2%, that is, the accuracy of Comparison case 1 is lower than that of Embodiment 1.

Although the invention is described in detail concerning the aforementioned embodiments, ordinary technicians in the field should understand that they can still modify the technical solutions recorded in the aforementioned embodiments, or replace some of the technical features equivalently; these modifications or replacements do not make the essence of the corresponding technical scheme separate from the spirit and scope of the technical scheme of each embodiment of the invention.

What is claimed is:

1. A method for determining multi-characteristic components of an Anshen Dingzhi Wan (ASDZW), comprising the following steps:
preparing a test solution of the ASDZW;
preparing a mixed reference solution by weighing polygalaxanthone III, 3,6′-disinapoyl sucrose, ginseng saponin Rb1, β-asarone, dehydrotumulosic acid, and pachymic acid;
detecting the test solution of the ASDZW and the mixed reference solution by a high performance liquid chromatography (HPLC) to obtain a liquid chromatogram of the test solution of the ASDZW and a liquid chromatogram of the mixed reference solution, respectively; and
comparing the liquid chromatogram of the test solution of the ASDZW with the liquid chromatogram of the mixed reference solution, and determining characteristic peaks and contents of the multi-characteristic components in the ASDZW according to a comparison result.

2. The method for determining the multi-characteristic components of the ASDZW according to claim 1, wherein the step of preparing the test solution of the ASDZW comprises:
performing an ultrasonic treatment for the ASDZW in a water-saturated n-butanol solution, taking a filtrate after filtrating, and evaporating to dry the filtrate to obtain a dried filtrate; and redissolving the dried filtrate with methanol and filtering by a microporous filter membrane to obtain the test solution of the ASDZW.

3. The method for determining the multi-characteristic components of the ASDZW according to claim 2, wherein a ratio of a mass of the ASDZW to a volume of the water-saturated n-butanol solution is 1: (15-20) g/mL; an ultrasonic treatment time is 20-40 min; and a pore size of the microporous filter membrane is 0.22-0.45 μm.

4. The method for determining the multi-characteristic components of the ASDZW according to claim 1, wherein the step of preparing the mixed reference solution comprises:
accurately weighting reference substances of the polygalaxanthone III, the 3,6′-disinapoyl sucrose, the ginseng saponin Rb1, the β-asarone, the dehydrotumulosic acid, and the pachymic acid, dissolving with methanol, and diluting to prepare the mixed reference solution.

5. The method for determining the multi-characteristic components of the ASDZW according to claim 1, wherein a concentration of the polygalaxanthone III in the mixed reference solution is 0.01-0.32 mg/mL; a concentration of the 3,6′-disinapoyl sucrose in the mixed reference solution is 0.03-1.00 mg/mL; a concentration of the ginseng saponin Rb1 in the mixed reference solution is 0.02-0.60 mg/mL; a concentration of the β-asarone in the mixed reference solution is 0.02-0.76 mg/mL; a concentration of the dehydrotumulosic acid in the mixed reference solution is 0.01-0.16 mg/mL; and a concentration of the pachymic acid in the mixed reference solution is 0.01-0.20 mg/mL.

6. The method for determining the multi-characteristic components of the ASDZW according to claim 1, wherein the step of detecting the test solution of the ASDZW and the mixed reference solution by the HPLC at 203 nm comprises the following steps:
determining test conditions of the HPLC and setting an operating parameter program of a gradient elution;
injecting the test solution of the ASDZW or the mixed reference solution into a sample valve; and
testing the test solution of the ASDZW or the mixed reference solution according to the operating parameter program.

7. The method for determining the multi-characteristic components of the ASDZW according to claim 6, wherein the test conditions of the HPLC comprise:
a mobile phase is an acetonitrile-phosphoric acid solution, wherein in the mobile phase, Phase A is acetonitrile and Phase B is a phosphoric acid solution; and a volume percentage concentration of the phosphoric acid solution is 0.09-0.11%, and
a flow rate of the mobile phase is 0.95-1.05 mL/min.

8. The method for determining the multi-characteristic components of the ASDZW according to claim 6, wherein the test conditions of the HPLC comprises:
a detector is a diode array detection (DAD) detector, and a detection wavelength is 203 nm;
a chromatographic column is a C18 chromatographic column, and a column temperature is 33-37° C.;
an injection volume is 10-20 μL; and an analysis time is 90 min, and the gradient elution is adopted.

9. The method for determining the multi-characteristic components of the ASDZW according to claim 8, wherein a filler in the chromatographic column is an octadecylsilane bonded silica gel.

10. The method for determining the multi-characteristic components of the ASDZW according to claim 8, wherein the operating parameter program of the gradient elution is as follows:
0-3 min, a volume percentage of Phase B is 85-84%, and a rest is Phase A;
3-5 min, the volume percentage of the Phase B is 84-78%, and the rest is the Phase A;
5-15 min, the volume percentage of the Phase B is 78%, and the rest is the Phase A;
15-18 min, the volume percentage of the Phase B is 78-75%, and the rest is the Phase A;
18-23 min, the volume percentage of the Phase B is 75-73%, and the rest is the Phase A;
23-27 min, the volume percentage of the Phase B is 73-67%, and the rest is the Phase A;
27-40 min, the volume percentage of the Phase B is 67-60%, and the rest is the Phase A;
40-45 min, the volume percentage of the Phase B is 60%, and the rest is the Phase A;
45-54 min, the volume percentage of the Phase B is 60-52%, and the rest is the Phase A;
54-59 min, the volume percentage of the Phase B is 52%, and the rest is the Phase A;
59-65 min, the volume percentage of the Phase B is 52-20%, and the rest is the Phase A; and
65-90 min, the volume percentage of the Phase B is 20%, and the rest is the Phase A.
